(19) United States
(12) Patent Application Publication
(10) Pub. No.: US 2007/0077311 A1
(43) Pub. Date: Apr. 5, 2007

(54) Fumigant/sterilant

(76) Inventors: Robert Francis Ryan, Sans Souci (AU); Simon Robert Bishop, North Ryde (AU)

Correspondence Address: THE BOC GROUP, INC., 575 MOUNTAIN AVENUE, MURRAY HILL, NJ, 07974-2064, US

(21) Appl. No.: 10/575,828

(22) PCT Filed: Oct. 15, 2004
(86) PCT No.: PCT/AU04/01417

§ 371 Date: Apr. 14, 2006

(30) Foreign Application Priority Data
Oct. 16, 2003 (AU) 2003905699

(51) Int. Cl. A61L 2/16

(52) U.S. Cl. 424/607; 422/32; 422/35

(57) ABSTRACT

A fumigant/sterilant comprising an effective amount of cyanogens mixed with a predetermined amount of carbon dioxide such that, in use, the fumigant/sterilant remains below its flammability limit. A method of producing said fumigant/sterilant is also provided.

### TECHNICAL FIELD

The present invention relates to a fumigant/sterilant, a method of producing such a fumigant/sterilant and a method of fumigation/sterilisation.

It is particularly suitable for post-harvest fumigation and sterilisation of pathogens in soil and/or stored commodities but it will be appreciated that the invention is not limited to this particular field of use.

### BACKGROUND OF THE INVENTION

Any discussion of the prior art throughout the specification should in no way be considered as an admission that such prior art is widely known or forms part of common general knowledge in the field.

Methyl Bromide is a well-known and widely used fumigant. However, this chemical has recently been listed on the Montreal Protocol as an ozone depletory and will not be available after 2005 in developed countries and after 2015 in developing countries. It is becoming imperative to find practical alternatives.

Other known chemical fumigants such as Methyl IsoThioCyanate, Telone®, Propylene Oxide and Methyl Iodide have been demonstrated as alternatives to Methyl Bromide as pre-plant and post-harvest fumigants. For example, Methyl Iodide (chemical formula CH₃I), also known as Iodomethane, is a liquid with a boiling point of 42° C. and is an effective soil fumigant for such crops as strawberries, vegetables, melons and nursery products. Similarly, the other above-mentioned Methyl Bromide alternatives are also available in liquid form.

Although the above list of fumigants is currently used as alternatives to Methyl Bromide in soil fumigation their use in confined space commodity fumigation is not well-known. There remains a need for a post-harvest stored product fumigant that has all the advantages of Methyl Bromide yet can be more easily and effectively applied during fumigation. Furthermore, a fumigant that can be conveniently packed in gas cylinders will make for simple substitution with the Methyl Bromide product.

It is an object of the present invention to overcome or ameliorate at least one of the disadvantages of the prior art, or to provide a useful alternative.

### DISCLOSURE OF THE INVENTION

According to a first aspect, the present invention provides a fumigant/sterilant comprising an effective amount of cyanogen mixed with a predetermined quantity of carbon dioxide such that in use, the fumigant/sterilant remains below its flammability limit.

The Applicants have found that such cyanogen (C₂N₂) is a potential Methyl Bromide alternative for fumigation/sterilisation and in particular for soil and stored commodities such as timber. Cyanogen is a very flammable liquefied gas and is reported to be subject to spontaneous combustion in air. While, in some cases, use of a flammable gas in soil and in stack fumigation of timber is an acceptable risk, its use in enclosed spaces, as required for commodity fumigation is a serious health and safety issue.

The Applicants have found, however, that it is possible to combine cyanogen with carbon dioxide such that in use it remains entirely below its flammability limit.

The liquefied flammable active chemical, ie cyanogen may be mixed with gaseous carbon dioxide in high pressure industrial gas cylinders as a prepackaged product, or may be mixed on site.

The fumigant preferably includes 1 to 26 wt % of cyanogen with the corresponding 99 to 72 wt % of carbon dioxide. In a further preferred form, the fumigant includes 1 to 20% cyanogen mixed with a corresponding 99 to 80% of liquid carbon dioxide.

Such a fumigant/sterilant will consistently remain below the flammability limit of cyanogen and is therefore suitable for a variety of uses including post-harvest fumigation and/or sterilisation of soil and commodities.

In addition to remaining below the flammability of cyanogen in air, formulating the active chemical with carbon dioxide improves the application and benefits of cyanogen by achieving superior dispensing, dispersion and efficacy in the fumigated commodities.

According to a second aspect, the present invention provides a method of producing a fumigant/sterilant comprising mixing an effective amount of cyanogen with a predetermined quantity of carbon dioxide such that in use, the fumigant/sterilant remains below its flammability limit.

As discussed above, such mixing can be achieved on site or alternatively, the method of fumigation may be accomplished by providing pre-packaged fumigants/sterilants comprising a high pressure cylinder of liquid cyanogen and liquid carbon dioxide in the desired quantities. Such a liquid cyanogen/carbon dioxide mix will, upon release, disperse into the atmosphere, act as an effective fumigant/sterilant and remain below the flammability limit of cyanogen in air.

According to a third aspect, the present invention provides use of a fumigant/sterilant of the first aspect, for fumigating and/or sterilising soil or commodities.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”.

### DETAILED DESCRIPTION OF THE INVENTION

A preferred embodiment of the present invention will now be described, by way of example only.

In the search for a suitable replacement of Methyl Bromide, the Applicants revisited the chemical cyanogen. As mentioned above, cyanogen is a very flammable liquefied gas and accordingly, it was necessary to determine if it was at all possible to provide a fumigant based on cyanogen which, in use, ie upon release, would pose a significant safety risk. Most fuel vapours and gases are only flammable or explosive at concentrations between their lower (LEL) and upper (UEL) explosion limits. These limits are normally determined by mixing known proportions of the fuel gas with fresh air, containing approximately 21% v/v oxygen. These mixtures are tested for propagation of flame after exposing the mixture to a suitable ignition source.

The flammability range of concentration tends to reduce as the oxygen content is reduced. The UEL and LEL approach each other and merge at an oxygen concentration beyond width propagation of an explosion is not possible for all proportions of fuel gas in the diluent gas. This oxygen concentration is referred to as the limiting oxygen concentration (LOC).

The Applicants proposed to determine whether it was possible to prepare a mixture of cyanogen in a suitable inert diluent gas such that when it is mixed with air, it remains below its LEL for all proportions while still remaining effective as a fumigant/sterilant. In use, the maximum amount of active chemical, ie cyanogen is preferred to increase efficacy of the fumigant/sterilant. However, this must be balanced against maintaining a safe operation level below the LEL.

Cyanogen is a flammable and highly poisonous gas with the following properties

Chemical formula—C₂N₂

Molecular weight—52.4 g/mol

Flammability limits in air—6-32% v/v

Exposure limits—TWA 10 ppm

LC₅₀ 350 ppm/1 hour inhalation—rat

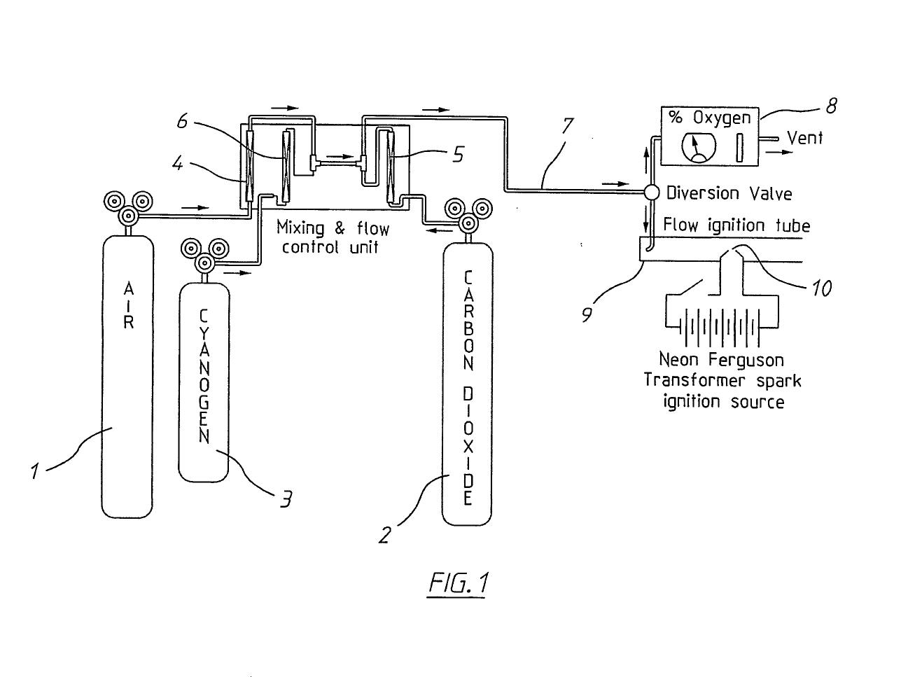

The apparatus for testing cyanogen flammability in air is shown in FIG. 1. The apparatus allows continuous metering and mixing of known proportions of cyanogen, carbon dioxide and air. This permitted a number of ignition tests to be made in a relatively short period.

The apparatus comprises sources of compressed air 1, carbon dioxide 2 and the active chemical cyanogen 3. Calibrated flow tubes 4, 5 and 6 respectively measure the flow rates of air, carbon dioxide and cyanogen and hence the composition of the mixtures. The resultant mixture flows through tube 7 where it is diverted to an oxygen analyser 8 to determine its content, and an ignition tube 9 with ignition source 10 for flammability testing.

Test Procedure

After suitable calibration of the cyanogen, air and carbon dioxide flow meters, flammability limit tests were conducted by introduction of a known flow of cyanogen gas into the known flow of air. Gas flow rates were altered to provide a range of values and at least two minutes were allowed to elapse for each adjustment of the flow and any ignition tests to ensure constant concentration.

Similarly, for the tests involving the addition of carbon dioxide, the proportions of air and CO₂ were set using the previously arrived calibration material and at least two minutes were allowed to elapse between adjustment of the CO₂/air proportions and ignition tests.

Ignition tests involved switching a high voltage across an approximately 5 mm gap 10 within the flow ignition tube 9. A test mixture was judged to be ignitable if a clear propagation of the flame away from the spark was observed. The resultant series of tests was used to “map” the limits of flammability of cyanogen in air, and the cyanogen/CO₂ mixtures in air.

By way of comparison, two ignition sources were tested to compare the LEL and UEL of the apparatus with the known LEL and UEL of cyanogen in air, ie 6% and 32% v/v.

Ignition source 1 found an LEL of between 7.5 and 8.1% and a UEL of 25.3 and 28.3%. Ignition source 2 found an LEL of between 5.8 and 6.6% and a UEL of 40.7 and 41.9.

It was determined that the flammability testing should be undertaken using ignition source 2 as this was more closely matched to the literature values for the LEL, ie the lower limit of explosability.

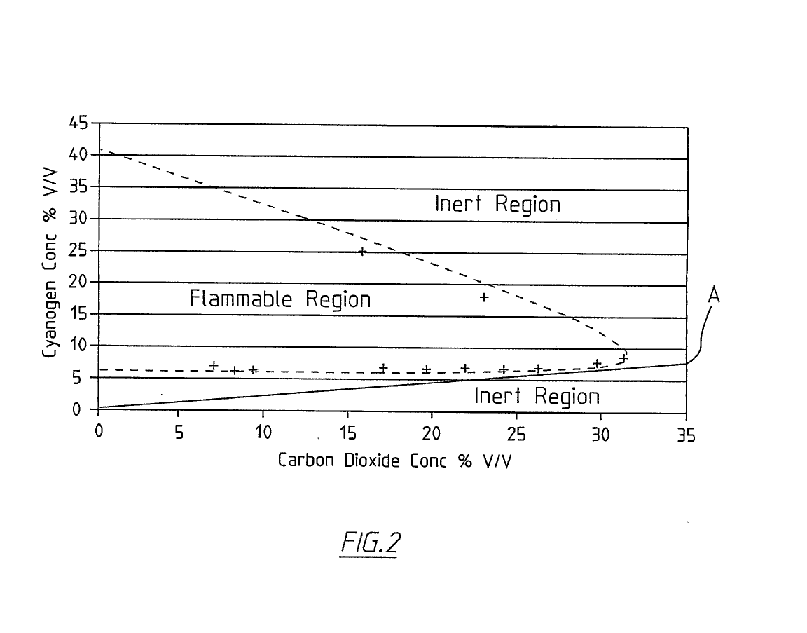

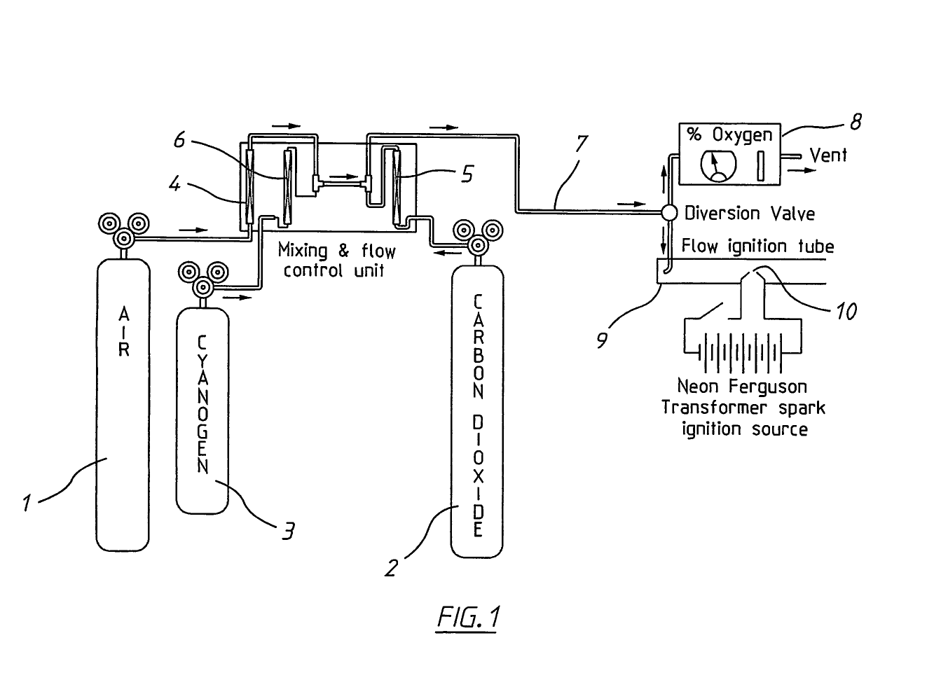

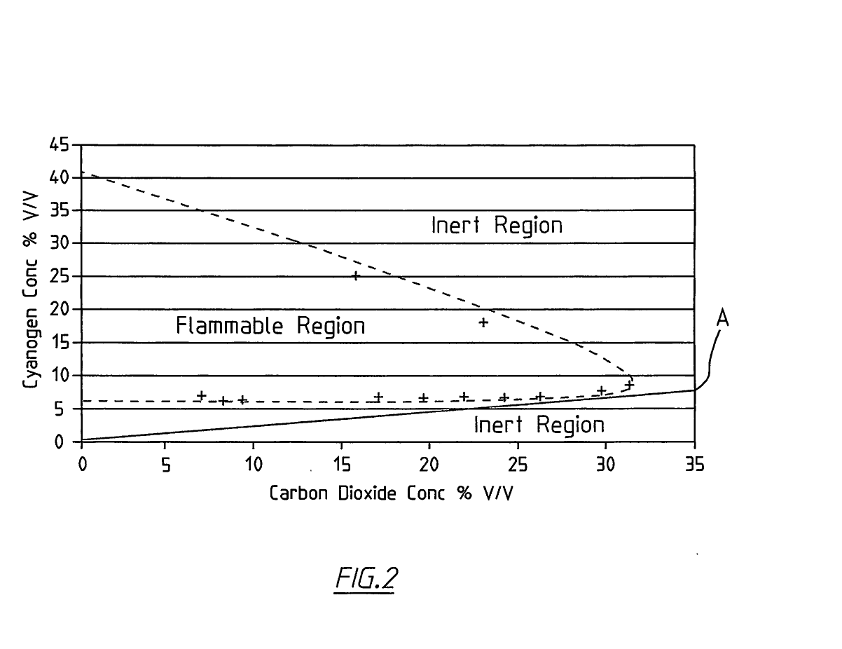

A large number of individual tests were conducted with varying cyanogen/CO₂/air contents. The resultant graph shown in FIG. 2 provides an accurate plot of the flammability limits for cyanogen/CO₂ mixtures in air. This plot shows the characteristic “nose” shaped zone of flammability and includes the practically determined LEL and UEL of cyanogen and carbon dioxide mixtures in air.

Turning to FIG. 2, it is now possible to determine what proportion of cyanogen in carbon dioxide will remain inert in all proportions with air. Line A shown in FIG. 2 has the maximum slope that can be achieved whilst still remaining wholly below the experimentally determined lower explosive limits and passing through the origin. This slope indicates that the maximum proportion of cyanogen in carbon dioxide which is inert in all proportions with air, ie approximately 26%.

Subsequent testing indicated that a maximum of around 26% v/v of cyanogen in carbon dioxide gas was inert in all proportions of air. For reasons of safety, the Applicant has determined a preferred content of around 20% v/v as this gives an additional margin of safety.

Accordingly, it can be seen that the present Applicants have developed a fumigant/sterilant comprising cyanogen and which in use will remain below its flammability limit in air in all proportions and still remain effective as a fumigant/sterilant.

The fumigant/sterilant of the invention is useful in a wide variety of environments. There are also many benefits of the flow from using carbon dioxide to dispense the cyanogen. Carbon dioxide provides the required pressure to spray the active chemical as required. In particular, the carbon dioxide supplies the force to dispense the mixture into confined gas tight spaces used for commodity fumigation/sterilisation. The carbon dioxide gas directs and disperses the liquid chemical and vaporises the liquid in space fumigation.

The use of carbon dioxide with cyanogen also improves efficacy of the cyanogen due to synergism. In particular, even at low levels, the Applicants have found carbon dioxide to be a synergist for many stored product fumigants and its reaction with moisture to form carbonic acid, also assists in the reduction of microbial levels, an issue in sterilisation.

Carbon dioxide enables the simple transport of the liquid fumigant from a container to a specific treatment zone. Of course, as discussed above, the cyanogen carbon dioxide may be mixed on site or placed into industrial gas cylinders. The thus resultant fumigant and method of fumigation provides a significant advance over conventional techniques. While it is a substitute for methyl bromide product and allows for easy substitution, it is not limited to such use.

It will be understood that the disclosed fumigant/sterilant, and method of production can be embodied in forms other than that described herein without departing from the spirit of scope of the invention.

1. A fumigant/sterilant comprising an effective amount of cyanogen mixed with a predetermined quantity of carbon dioxide such that in use, the fumigant/sterilant remains below its flammability limit.

2. The fumigant/sterilant according to claim 1, wherein said cyanogen is in the form of a liquefied gas.

3. The fumigant/sterilant according to claim 1, wherein said carbon dioxide is gaseous carbon dioxide.

4. The fumigant/sterilant according to claim 1, wherein said carbon dioxide is liquid carbon dioxide.

5. The fumigant/sterilant according to claim 1 comprising 1 to 26 wt % of cyanogen and 99 to 72 wt % of carbon dioxide.

6. The fumigant according to claim 5, comprising 1 to 20% cyanogen and 99 to 80% of carbon dioxide.

7. The fumigant/sterilant according to claim 1, wherein said cyanogen is mixed with said carbon dioxide on site or as a prepackaged product.

8. The fumigant/sterilant according to claim 1, wherein said cyanogen is mixed with said carbon dioxide in an industrial gas cylinder.

9. A method of producing a fumigant/sterilant comprising mixing an effective amount of cyanogen with a predetermined quantity of carbon dioxide such that in use, the fumigant/sterilant remains below its flammability limit.

10. The method according to claim 9, wherein said cyanogen is in the form of a liquefied gas.

11. The method according to claim 9, wherein said carbon dioxide is gaseous carbon dioxide.

12. The method according to claim 9, wherein said carbon dioxide is liquid carbon dioxide.

13. The method according to claim 9, wherein 1 to 26 wt % of cyanogen is mixed with 99 to 72 wt % of carbon dioxide.

14. The method according to claim 13, wherein 1 to 20% cyanogen is mixed with 99 to 80% of carbon dioxide.

15. The method according to claim 9, wherein said cyanogen is mixed with said carbon dioxide on site or as a prepackaged product.

16. The method according to claim 9, wherein said cyanogen is mixed with said carbon dioxide in an industrial gas cylinder.

17. Use of the fumigant/sterilant according to for fumigating and/or sterilising soil or commodities.

18. Use of the fumigant according to claim 17, for post-harvest fumigation and/or sterilisation.

19. Use according to claim 17, wherein said commodity is timber.

20. Use of the fumigant/sterilant according to claim 1, for confined space soil or commodity fumigation and /or sterilisation.
